(19) United States
(12) Patent Application Publication
(10) Pub. No.: US 2002/0048755 A1
(43) Pub. Date: Apr. 25, 2002

(54) System for developing assays for personalized medicine

(76) Inventor: Jonathan M. Cohen, Potomac, MD (US)

Correspondence Address: Henry N. Wixon, HALE AND DORR LLP, 1455 Pennsylvania Avenue N.W., Washington, DC, 20004, US

(21) Appl. No.: 09/769,298

(22) Filed: Jan. 26, 2001

Related U.S. Application Data
(60) Provisional application No. 60/178,222, filed on Jan. 26, 2000.

(51) Int. Cl. C12Q 1/68; G01N 33/48

(52) U.S. Cl. 435/6; 435/7.21; 435/960; 435/40.5; 435/40.52; 424/9.1; 424/9.2; 424/9.34; 514/44

(57) ABSTRACT

A system for developing diagnostic assays, useful in determining whether a particular therapeutic agent will benefit an individual, comprises a continuum of processes that advance diagnostic development while concomitantly benefiting development of the therapeutic agent. This continuum of processes that are dual use, in promoting both diagnostic and drug development, is highly economical and efficient, and creates synergy between pharmaceutical and diagnostic companies.

### FIELD OF THE INVENTION

The present invention is directed to a system for developing target specific assays for determining whether a patient will likely respond to a target specific drug, and more particularly to a such a system that is highly economical and provides synergies when diagnostics and drugs are developed in parallel.

### BACKGROUND OF THE INVENTION

Once the human genome has been sequenced a key challenge will be the identification from among the more than 100,000 human genes valid therapeutic targets, molecules with which a drug can be designed to interact and produce a therapeutic effect. For such an effort it will likely be desirable to have diagnostic assays specifically designed to detect the target that can be used both in a research setting to validate the target and thereafter in a clinical setting to help guide in the selection of patients to receive the drug.

Unfortunately, drugs and diagnostics are typically developed independently of one another and few companies will have an incentive to develop diagnostics linked to particular drugs since such tests are typically administered only once making it difficult to recoup the considerable investment required for diagnostic development. Moreover, assays developed for research applications—such as target validation—are rarely developed with the thought of eventually commercializing the assay. These tests, often referred to as “home brew” assays, are typically designed for research use only. This is problematic since tests performed in a hospital or regional reference laboratory have different requirements from those done in a research setting. For example, in a clinical setting where large volumes of samples are received each day from numerous patients tests need to be designed to be run on an automated instrument. In a research laboratory manual assays are more commonplace. Conversion of a manual diagnostic into one for an automated platform is often time consuming and expensive especially when the research lab has no relationship with the commercial diagnostic company and biological materials and information is not passed to the commercial manufacturer.

The aforementioned challenges can be understood by considering the process that led to the development of HERCEPTIN® (Genentech, S. San Fransisco, Calif.) among the first approved target-specific drugs with a target-specific diagnostic linked thereto. A description of the development of this drug is set forth in the book HER-2, Random House, New York 1998.

In the mid 1980s researchers evaluated tissue samples from almost 200 primary breast cancers for alterations in the HER-2 oncogene which encodes a receptor having tyrosine kinase activity. The tissues used in this study included patient outcomes. As disclosed in U.S. Pat. No. 4,968,603 to Slamon et al., the researchers discovered a correlation between amplification of that gene and time to disease relapse and survival. Approximately 25-30 percent of women with breast cancer have cancers that overexpress the HER-2 oncogene, which is associated with more rapid cancer progression. Because of the correlation found between overexpression and disease outcome the researchers deemed the HER-2 gene a “logical target” for therapy. HER-2 at 185. This led to the development of HERCEPTIN® by the company that the researchers were associated

HERCEPTIN® is a monoclonal antibody that targets metastatic breast cancer cells that overexpress the HER-2 oncogene. HERCEPTIN® works by binding to the HER-2 growth factor receptors present in excessive amounts on the surface of the cancer cells. The drug is indicated only for patients whose tumors have either amplification (i.e. extra copies) of the HER-2 gene as determined by an in-situ hybridization (ISH) assay or protein overexpression as determined by an immunohistochemistry (IHC) assay. HER-2 status has also been found to predict patient response to a variety of conventional therapeutic agents such as doxorubican.

Before a drug or diagnostic product can be marketed in the United States and most other countries it is subjected to strict regulatory review of its safety and efficacy. In the case of a diagnostic for personalized medicine this will likely require the testing of tissue or bodily fluids from patients that received the drug to ascertain whether there is a link between their response to therapy and the presence of a particular target such as an overexpressed or truncated protein. Once the diagnostic has been shown effective in predicting patient response, if there is any change in any characteristics of the diagnostic to be sold from the one used on the clinical studies, such as the sequence of the probe (or specificity of the antibody), the test protocol (time, temperature, reaction condition) of format of the assay (manual vs. automated) a new clinical study is usually required.

In the case of HERCEPTIN®, the safety and efficacy were studied in clinical trials of patients having metastatic breast cancer whose tumors overexpress the HER-2 protein as measured by an IHC research-use-only assay of tumor tissue performed by a reference laboratory. Patients were eligible to participate in the trial if they had 2+ or 3+ levels of overexpression (based on a 0-3+ scale) by IHC assessment of tumor tissue performed by at the research lab. Data from the trials suggested that the beneficial treatment effects were largely limited to patients with the highest level of HER-2 protein overexpression.

Because the test used during the HERCEPTIN® drug trials was a “home brew” assay not designed by a company that normally sells diagnostics, the specifics of the test (e.g. protocol, reagent concentrations, features for use with an automated instrument) were designed only with the drug trial in mind rather than ultimately commercializing the test. It later became apparent, however, that if a diagnostic was used to guide patient selection during clinical trials then the diagnostic, or its equivalent, would need to be available after the drug is approved for marketing. Thus a need quickly arose for a commercial version of the research diagnostic used during the drug trial. However, as stated, before such a diagnostic can be sold it must be tested in clinical studies that establish the ability of the diagnostic to determine which patients are more likely to benefit from the drug. If there is any change in material properties of the diagnostic to be sold from the one used in the clinical studies, such as the sequence of the probe (or specificity of the antibody), the test protocol (time, temperature, reaction condition) of format of the assay (manual vs. automated) a new clinical study is usually required. Subsequently, after the drug trials were concluded, several companies sought regulatory approval to market IHC tests that detect HER-2 expression to determine whether patients are eligible to receive HERCEPTIN®. To do so these companies had to prove, to the satisfaction of regulatory authorities, that their commercial assay was equivalent to the research assay that was used in the clinical trials of the drug HERCEPTIN®. This process was time consuming and expensive. For example, one company had to compare the results of its IHC assay with the research assay used in the clinical trials on over 500 breast cancer specimens. Furthermore, even after the commercial assays were approved they could not be legally marketed without a warning label that read “the actual correlation of the diagnostic to the drug's clinical outcome has not been established.” Such a warning clearly has negative marketing implications.

In sum, in the development of HERCEPTIN® required large collections of diseased tissue had to be screened for gene amplification/overexpression three times: (i) during the research phase to correlate gene amplification with disease outcome, (ii) in the validation of the clinical trial assay, and (iii) in the development and approval of the commercial diagnostic to prove equivalency to the clinical trial assay. This is unfortunate since human disease tissue is a scarce commodity, especially samples with reports detailing the medical histories of the patient from whom the tissue was excised.

It would therefore be desirable to have a system for developing diagnostics which permitted more conservation of human disease tissue.

It would also be desirable to have a system that avoids the time and expense of proving equivalency between the diagnostic used in a drug trial and one used in the marketplace by testing the commercial diagnostic in parallel with the drug so as to allow the drug and diagnostic to go through clinical trials in tandem.

It would also be desirable to avoid duplication of effort by using the same assay during the research phase to establish or validate targets in both clinical trials and in the marketplace.

### SUMMARY OF THE INVENTION

The present invention is directed to a system for developing diagnostic assays for determining whether a particular therapeutic agent will benefit an individual. The system comprises a continuum of processes that advance diagnostic development while at the same time benefitting the entity developing the therapeutic agent. This continuum of “dual use” processes (i.e. processes that benefit both diagnostic and drug development) has the particular advantage in that it is highly economical, expeditious, efficient, and creates synergies between pharmaceutical and diagnostic companies.

The continuum of processes according to the present invention preferably comprises three distinct phases: (i) target validation (i.e., establishing the clinical utility of a macromolecule as a target of therapy) by developing an assay to screen for the target in large quantities of tissues from different patients, organs, diseases, or disease stages, (ii) using the assay to select patients in a clinical trial to test the efficacy of a drug designed to interact with the target while at the same time testing the effectiveness of the assay, and (iii) using the assay in the marketplace to help determine whether a target specific drug should be prescribed to a particular patient based on the characteristics of the target in tissue removed from the patient.

It is a particular advantage of the present invention that many of the efforts employed to develop an assay in one phase need not be repeated in subsequent phases. For example, an antibody that is raised and optimized to bind to a specific target can be used in the target validation, clinical trial and marketplace phases. Similarly, the protocol for in-situ hybridization, which often takes a great deal of time and effort to develop, can be “recycled” for use in subsequent phases (see Table 1). This avoids unnecessary duplication of efforts.

Another key advantage of the system according to the present invention is that the efforts at each phase benefit both drug and diagnostic development. For example, the assay created for target validation helps drug developers ascertain the relevance of the target for therapy and may also be a useful diagnostic product in its own right. Furthermore, an assay used to select patients during a clinical trial may not only help expedite drug approval but, if designed and used in a particular manner, can latter be sold commercially as a diagnostic with few regulatory barriers to overcome. In the case of target validation, for each tissue sample the quantity or location of target is determined and compared to other samples from different organs or from patients in different disease states. For example, determining that amplification or overexpression of a particular gene is more frequent in tumors from patients with a recurrent form of cancer may create a prognostic marker used in planning treatment strategies as well as a target for designing new drugs that interact with the gene or its product. Thus, each phase provides a “dual use” function that permits some of the costs of diagnostic development to be shifted to the pharmaceutical companies which typically have greater resources.

Yet another advantage of the present invention is the speed and high-throughput achieved through the use of the combination of tissue microarrays together with the automated staining instrumentation.

Still another advantage of the present invention is that it allows accurate comparison of results from multiple different tissue samples each having been treated in precisely the same manner.

Yet another advantage of the present invention is that the same staining protocol (reagents, times, temperatures, etc.) developed for evaluating or validating a target in a research setting can be subsequently employed a clinical (patient care) setting for disease prognosis or treatment selection.

With the foregoing and other objects, advantages and features of the invention that will become hereinafter apparent, the nature of the invention may be more clearly understood by reference to the following detailed description of the invention, the appended claims and to the several views illustrated in the drawings.

### BRIEF DESCRIPTION OF THE DRAWINGS

FIG. 1 is a schematic illustration showing the system for assay development according to the present invention.

FIG. 2 is a schematic illustration of the target validation method according to the present invention.

### DETAILED DESCRIPTION OF THE INVENTION

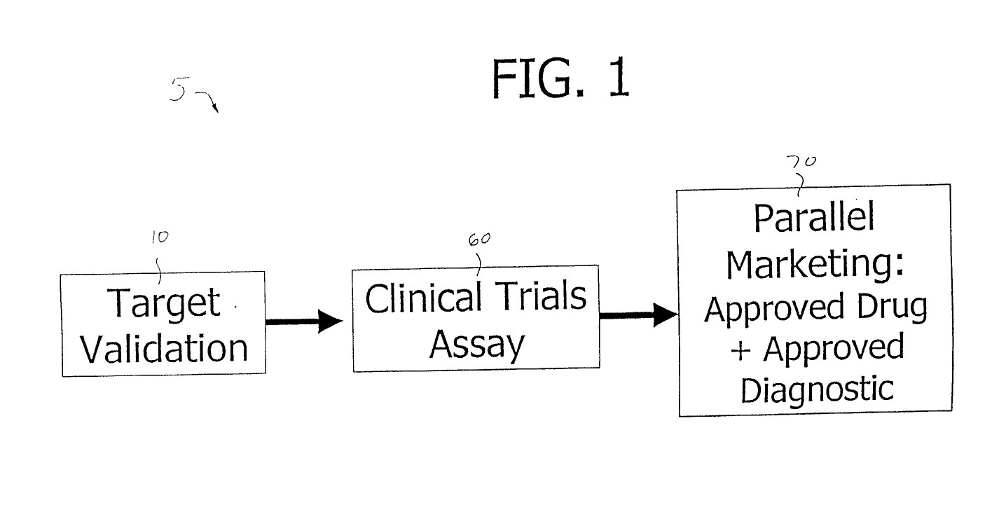

Referring now in detail to the drawings wherein like parts are designated by like reference numerals throughout, there is illustrated in FIG. 1 a schematic illustration showing the system for assay development according to the present invention which is designated generally by reference numeral 5. System 5 generally comprises a continuum of processes that perform the dual functions of providing a valuable service to companies that are developing drugs while at the same time contributing to the development of commercial diagnostics for use in a clinical setting.

The continuum of processes according to the present invention preferably comprises three distinct phases: (i) target validation 10 (i.e., establishing the clinical utility of a macromolecule as a target of therapy) by developing an assay to screen for the target in large quantities of tissues from different patients, organs, diseases, or disease stages, (ii) clinical trials assay 60 (i.e. using the assay to select patients in a clinical trial to test the efficacy of a drug designed to interact with the target while at the same time testing the effectiveness of the assay), and (iii) parallel marketing 70 (i.e., using the assay in the marketplace to help determine whether a target specific drug should be prescribed to a particular patient based on the characteristics of the target in tissue removed from the patient).

Each of the aforementioned phases of system 5 will now be described in more detail.

### Definitions

The following terms shall have the following meanings as used herein:

“Automated” or “Automatic” means activity substantially computer controlled or machine driven and substantially free of human intervention during normal operation.

“Clinical Utility” means usefulness of a target for (i) designing or prescribing a drug or therapy that interacts with the target, or (ii) determining which patients would be most likely to benefit from a particular drug or therapy.

“Different Tissue” means tissue from different patients, organs, diseases, and/or disease stages.

“High-Throughput” means the capability to treat more than about 20,000 different tissue samples in one day with one operator.

“Sources” and “Target Sources” means companies or similar entities that provide the system according to the present invention with at least one target, receive services from the system, and are separately controlled from the company that uses the system.

“Screen” means determining the presence, absence, quantity, location, and/or other characteristics of a target in a tissue sample.

“Stain” means any biological or chemical substance which, when applied to targeted molecules in tissue, renders the molecules detectable under a microscope. Stains include without limitation detectable nucleic acid probes, antibodies, and dyes.

“Target” and “Targeted molecules” means detectable molecules found in cells including without limitation nucleic acids, proteins, antigens, carbohydrates, lipids, and small molecules.

“Tissue” means any collection of cells that can be mounted on a standard glass microscope slide including, without limitation, sections of organs, tumor sections, bodily fluids, smears, frozen sections, cytology preps, and cell lines.

“Tissue Array” and “Tissue Micorarray” means a glass microscope slide or similar solid surface having a plurality of different tissue samples thereupon.

“Treat”, “Treating” or “Treatment” shall mean application of a stain to a tissue as well as other processes associated with such application including, without limitation, heating, cooling, washing, rinsing, drying, evaporation inhibition, deparaffinization, cell conditioning, mixing, incubating, and/or evaporation.

“Validation” or “Target Validation” means screening tissues in order to confirm the relevance of a potential target for action by a therapeutic.

### 1. Target Validation

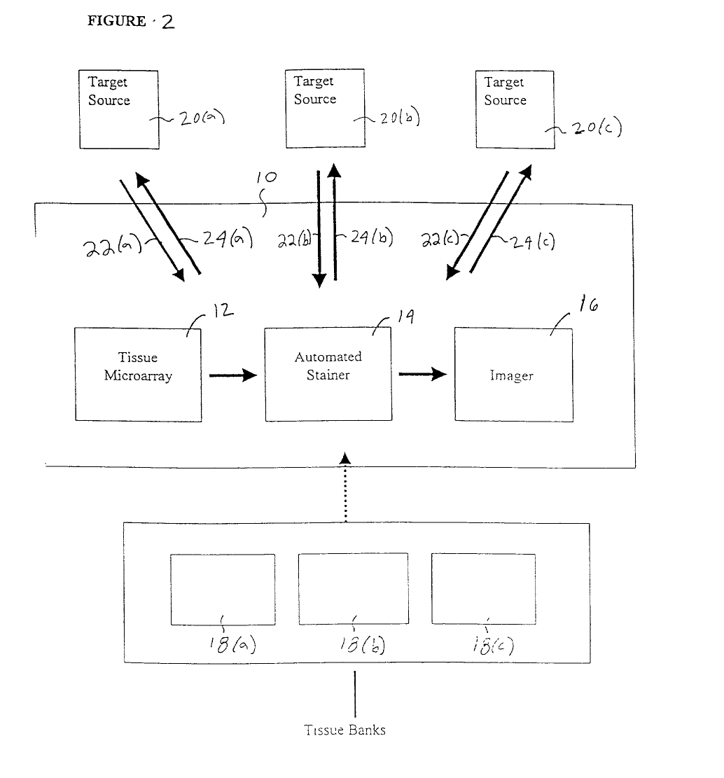

With reference to FIG. 2 the target validation phase 10 is substantially as described in U.S. Provisional Application No. 60/155,665 filed Sep. 24, 1999 which is incorporated herein in its entirety. In short phase 10 generally utilizes tissue microarray apparatus 12 for constructing arrays of hundreds of minute tissue samples mounted on a single glass microscope slide, staining apparatus 14 for automatically conducting most of the steps required for ISH/IHC, and imaging apparatus 16 to allow the results of the ISH/IHC staining to be visualized and analyzed by the user. System 10 preferably has access to one or more tissue banks 18 (a-c) having thousands of preserved surgical samples catalogued by organ type, disease, and patient history. In use and operation system 10 is adapted to serve multiple sources of different targets 20 such as pharmaceutical companies and the like who each supply the system with one or more molecular targets 22 (DNA, RNA, or protein). and receive data 24 regarding the clinical relevance of the targets based on screening of the tissue samples assayed.

Sources 20 of target molecules for system 10 would include pharmaceutical and biotechnology companies, that have identified novel targets believed to be associated with a particular disease or disorder including genes, gene fragments, mRNA sequences, or antigens. Typically they have an idea or prediction of the targets' biological function from profiling the expression pattern of clinical samples using one or more technologies such as sequence homology, Northern blot, SAGE or DNA microarrays.

With this data the user of system 10 would access tissue banks 18 and select between 30 and 1000 blocks representing different patient populations and disease states. The selected blocks are used as donor blocks. The types of tissue samples selected would depend largely on the diseases for which new in situ assays would be deemed useful in medical practice. This would include cancer, ostoarthritis, rheumatoid arthritis, asthma, and skin disorders such as psoriasis and eczema. This might also include tissues from patients diagnosed Chron's disease, type I diabetes, and certain other autoimmune disorders.

Sections cut from the array allow parallel detection of DNA (fluorescense in situ hybridization, FISH), RNA (mRNA ISH) or protein (immunohistochemistry, IHC) targets in each of the hundreds of specimens in the array. Preferably staining instrument 14 is employed to carry out the staining protocols in an automated manner. Alternatively, manual staining of the microarray may first be employed followed by automatic staining of conventional samples with instrumentation 14 to confirm the results of the array. For some diseases (e.g. osteoarthritis) conventional sections will need to be used in lieu of the minute samples used with arrays as will be readily apparent to one of skill in the art.

Staining instrument 14 may be used to perform in-situ hybridization (ISH), in-situ PCR, immunohistochemistry (IHC), Special Stains; as well as a variety of chemical (non-biological) tissue staining techniques on an array or conventional tissue specimens. Moreover, two or more of the above techniques may be employed during a single run despite their differing temperature requirements due to the inventive heating system herein.

The stained slides would be scored and analyzed by a pathologist or pathology support personnel using techniques known in the art. The results would be preferably be correlated by a biostatistician to arrive at clinical utility of the target in tissue. For example, it might be determined that overexpression of the gene target is a particular tumor type correlates with extended survival in patients treated with a drug designed to block expression of the gene target. A useful in situ assay could then be developed for use in selecting patients to receive the drug.

System 10 should be capable of screening large volumes of tissue samples in a high-throughput manner. If both tissue microarray 12 and automated staining instrumentation 14 are used at least one run and perhaps two runs of twenty slides, each supporting up to 1000 minute tissue samples may be treated in one day with a single operator. Thus between 20,000 and 40,000 different samples may be screened per day with a single operator using system 10.

### 2. Assays Used in Drug Trials

After the target of therapy has been validated a drug is selected or designed to specifically block or enhance the activity of the targeted molecule. If the target is an enzyme the drug may be an inhibitor of the enzyme. If the target is a cellular receptor the drug may be an agonist or antagonist to the receptor.

In most countries drugs must be proven safe and effective for their intended use before they can be marketed. This usually involves extensive human clinical trials. In order to select patients most likely to respond to the target-specific drug it is often desirable to determine the quantity or structure of the target in tissue samples removed from the patient. For example, if the target is a growth factor receptor involved in malignancy it may be desirable to stain biopsy samples with an IHC antibody specific for the receptor it order to determine overexpression of the receptor. In addition to IHC, other in-situ techniques such as ISH, PRINS, and in-situ PCR may be employed in order to determine both the degree and location of overexpression.

In the continuum of processes (FIG. 1) according to the present invention a clinical trial assay 60 is developed following target validation 10. Preferably, assay 60 utilizes many, if not all, of the reagents and protocol developed during the target validation phase. These generally include, without limitation, the primary antibody (IHC) or nucleic acid probe (ISH), labeling scheme (fluorescent or Brightfield) and the particular hapten used for labeling (e.g. digoxigenin) and optimized staining protocol for automated instrumentation (incubation time, hybridization temperatures, reagent concentrations, etc.).

It is a particular feature of the present invention that the drug and diagnostic are tested together in the same trial so that the effectiveness of the diagnostic can be tested on tissue samples from patients seeking to be enrolled in the trials. To further reduce the quantity of tissue and time required a tissue microarray as described in U.S. Provisional Application No. 60/155,665 may be employed so that minute samples from hundreds of patients can be treated simultaneously. This “trial on a chip” approach can significantly reduce time and other resources.

If a link between the presence of the target and response to therapy has been conclusively established through in-vtro studies, animal models, retrospective analyses, and the like then the diagnostic will be used to select patients for enrollment at the outset of the first phase of the drug trial for which efficacy is being tested (typically phase II). On the other hand, if the effectiveness of the diagnostic as a predictor of response to therapy has not been proven to the satisfaction of regulatory authorities or the sponsors of the trials it may be desirable to initially enroll patients regardless of gene status and determine during the trial if a clear correlation emerges between response to therapy and overexpression or mutation of the target genes.

### 3. Parallel Marketing of Drug and Diagnostic

It is a particular feature of the present invention that the clinical trial assay was designed with the view that it will be ultimately marketed to pathology labs in hospitals and other clinical reference laboratories. Reagent labeling is preferably brightfield labeled to be compatible the light microscopes in most pathology labs. The protocol is preferably suitable for an automated instrument such as the DISCOVERY instrument sold by Ventana Medical Systems, Inc. (Tucson, Ariz.). Preferably the company that designed and manufactured the clinical trials assay will also make and sell the commercial version of the diagnostic. This will avoid the time and expense of having to run another study to prove equivalency etc. thereby consuming more human tissue samples which is, as stated, a scarce resource. It also avoids the need to transfer biological materials and data between organizations with differing operating procedures.

The economic advantages of using the same test for target validation, drug trials, and commercial diagnostic development are set forth in the following Table 1.

Although certain presently preferred embodiments of the invention have been described herein, it will be apparent to those skilled in the art to which the invention pertains that variations and modifications of the described embodiment may be made without departing from the spirit and scope of the invention. Accordingly, it is intended that the invention be limited only to the extent required by the appended claims and the applicable rules of law. The references cited above are hereby incorporated herein in their entirety.

TABLE 1
Economic Advantages of Continuum of Processes

| | | Assay | Commercial |
|---|---|---|---|
| | Target | Development | Diagnostic |
| TASK: | Validation | for Drug Trials | Development |
| Procure archival tissues w/ | Yes | ✓ | ✓ |
| clinical follow-up for | | | |
| retrospective Studies | | | |
| Disease tissue ID | Yes | ✓ | ✓ |
| by pathologist | | | |
| Create tissue microarray | Yes | ✓ | ✓ |
| Raise/Select | Yes | ✓ | ✓ |
| Primary Antibody | | | |
| Synthesize/Optimize Probe | | | |
| and labeling scheme | | | |
| Select Detection System | Yes | ✓ | ✓ |
| (Brightfield for ultimate | | | |
| use in Pathology Labs) | | | |
| Optimize staining conditions | Yes | ✓ | ✓ |
| for automated instrumenta- | | | |
| tion (incubation time, temper- | | | |
| ature, concentration, etc.) | | | |
| Stain tissues using automatic | Yes | ✓ | ✓ |
| stainer w/ multiple probes, | | | |
| Abs | | | |
| Interpret expression, | Yes | ✓ | ✓ |
| detection, or amplification | | | |
| patterns; review by pathol- | | | |
| ogist; statistical correlation | | | |
| GMP reagent manufacturing | N/A | ✓ | ✓ |
| and scale-up | | | |
| Retrospective/Prospective | N/A | ✓ | ✓ |
| clinical study using tissue | | | |
| from patients enrolled in | | | |
| drug trial (multi-center) | | | |
| Submission of IDE w/FDA | N/A | N/A | ✓ |

✓ = step completed in earlier phase and need not be repeated

What is claimed is:

1. A method for testing the safety and efficacy of both a drug and a diagnostic assay linked to the drug comprising the steps of:
A. Providing a drug;
B. Providing an ISH or IHC diagnostic assay for selecting a population of patients to receive said drug;
C. Conducting a clinical trial by administering said drug to said population of patients selected using said diagnostic assay;
D. Wherein data regarding the safety and efficacy of both the drug and the diagnostic assay used in the clinical trial are submitted to a regulatory authority following the clinical trial.

2. A diagnostic assay tested according to the method of claim 1.

3. A drug tested according to the method of claim 1.

4. A method for developing a diagnostic assay for use in personalized medicine comprising the steps of:
A. Providing a target;
B. Providing an ISH or IHC assay to detect said target in tissue samples;
C. Validating said target in a plurality of tissues;
D. Providing a drug to interact with said target;
E. Using said assay to detect the quantity of said target in tissue samples removed from a patient so as to identify whether said patient would likely benefit from said drug.

5. A method for developing an ISH or IHC diagnostic kit for selecting patients to receive a drug comprising the steps of:
A. Selecting the optimal reagents and protocol for said diagnostic kit;
B. Conducting a clinical trial of a drug using said diagnostic kit with said reagents and protocol; and
C. Making and selling said diagnostic kit with substantially the same reagents and protocol used in said clinical trial.
